(19) United States
(12) Patent Application Publication
(10) Pub. No.: US 2016/0038618 A1
(43) Pub. Date: Feb. 11, 2016

(54) PLASMONIC SUB-100 NM NANOMATRYOSHKAS THAT CONFINE CONTRAST AGENTS WITHIN LAYERS OF METAL

(71) Applicant: William Marsh Rice University, Houston, TX (US)

(72) Inventors: Nancy J. Halas, Houston, TX (US); Ciceron Ayala-Orozco, Stafford, TX (US); Sandra Bishnoi, Houston, TX (US)

(21) Appl. No.: 14/823,891

(22) Filed: Aug. 11, 2015

Related U.S. Application Data
(60) Provisional application No. 62/035,716, filed on Aug. 11, 2014.

(51) Int. Cl. A61K 49/18

(52) U.S. Cl. CPC A61K 49/1878

(57) ABSTRACT

A magnetic resonance imaging enhancement agent includes a plurality of particles. Each particle including a metal core; a dielectric shell disposed on the metal core including water and at least one MRI contrast agent; and a metal shell disposed on the exterior surface of the dielectric shell that encapsulates the dielectric shell.

### CROSS-REFERENCE TO RELATED APPLICATIONS

This application is a non-provisional patent application of U.S. Provisional Patent Application Ser. No. 62/035,716, filed on Aug. 11, 2014, and entitled: “Plasmonic sub-100 nm nanomatryoshkas that include contrast agents within layers of metal.” Accordingly, this non-provisional patent application claims priority to U.S. Provisional Patent Application Ser. No. 62/035,716 under 35 U.S.C. §119(e). U.S. Provisional Patent Application Ser. No. 62/035,716 is hereby incorporated in its entirety.

### STATEMENT REGARDING FEDERALLY SPONSORED RESEARCH OR DEVELOPMENT

The invention was made with government support under Grant Number U01 CA151886 awarded by the National Institutes of Health. The government has certain rights in the invention.

### BACKGROUND

In magnetic resonance imaging, there are two types of contrast agents: type 1 (T1) and type 2 (T2). T1 agents are positive contrast agents that make an image brighter on MRI phantoms. T2 agents are negative contrast agents that cause a darker image on MRI phantoms. Contrast agents for MRI lighten or darken MRI phantoms by modifying the relaxation time of the spins of protons in water. Commercial T1 contrast agents need to be in direct contact with water to produce its effect while T2 agents not need to be in direct contact with water.

### SUMMARY

In one aspect, magnetic resonance imaging enhancement agent according to one or more embodiments may include a plurality of particles, each particle including a metal core; a dielectric shell disposed on the metal core including water and at least one MRI contrast agent; and a metal shell disposed on the exterior surface of the dielectric shell that encapsulates the dielectric shell.

In one aspect, a method of producing a magnetic resonance imaging enhancement particle may include coating a metal core with a dielectric to obtain a metal core with a dielectric coating; loading the dielectric coating with a solution including water and a MRI contrast agent to obtain a loaded dielectric coating; etching the loaded dielectric coating to a desired thickness to obtain an etched dielectric coating; seeding the exterior of the etched dielectric coating with a metal to obtain a seeded dielectric coating; and coating the seeded dielectric coating with a second metal to obtain the magnetic resonance imaging enhancement particle.

### BRIEF DESCRIPTION OF DRAWINGS

FIG. 1 shows a cross sectional view of a single particle of a magnetic resonance imaging contrast enhancement agent in accordance with one or more embodiments.

FIG. 2A shows a step of a method of producing a magnetic resonance imaging enhancement agent in accordance with one or more embodiments.

FIG. 2B shows a step of a method of producing a magnetic resonance imaging enhancement agent in accordance with one or more embodiments.

FIG. 2C shows a step of a method of producing a magnetic resonance imaging enhancement agent in accordance with one or more embodiments.

FIG. 2D shows a step of a method of producing a magnetic resonance imaging enhancement agent in accordance with one or more embodiments.

FIG. 2E shows a step of a method of producing a magnetic resonance imaging enhancement agent in accordance with one or more embodiments.

FIG. 3A shows a transmission electron microscope (TEM) image of a plurality of particles of a magnetic resonance imaging contrast enhancement agent in accordance with one or more embodiments.

FIG. 3B shows an extinction spectrum of a plurality of particles of a magnetic resonance imaging contrast enhancement agent in accordance with one or more embodiments.

FIG. 3C shows the T1 enhancement of a plurality of particles of a magnetic resonance imaging contrast enhancement agent in accordance with one or more embodiments.

FIG. 3D shows the relaxivity of a plurality of particles of a magnetic resonance imaging contrast enhancement agent in accordance with one or more embodiments.

### DETAILED DESCRIPTION

Specific embodiments will now be described in detail with reference to the accompanying figures. In the following description, numerous details are set forth to provide an understanding of the present disclosure. However, it will be understood by those skilled in the art that the embodiments of the present disclosure may be practiced without these details and that numerous variations or modifications from the described embodiments may be possible.

In general, embodiments of the invention relate to a magnetic resonance imaging contrast enhancement agent comprising a plurality of particles with properties to enhance magnetic resonance imaging and photothermal ablation. Further, embodiments of the invention may combine the aforementioned magnetic resonance imaging enhancement agent with antibody and/or peptide targeting and/or photothermal therapeutic actuation.

In one or more embodiments of the invention, antibody targeting may be used such that the magnetic resonance imaging contrast enhancement agent may bind to the surface receptors of specific cell types. In the case of cancer therapy, the magnetic resonance imaging contrast enhancement agent may allow for the tracking the location of the particles in vivo. For example, magnetic resonance imaging may be used to follow the path of the particles or verify the quantity of particles at specific locations. Once verified, ablation of the targeted cells may be carried out by photothermal ablation.

In one or more embodiments of the invention, the magnetic resonance contrast enhancement agent may be used in other medical imaging techniques including Positron Emission Tomography-Computed Tomography (PET-CT), Positron Emission Tomography-Magnetic Resonance Imaging (PET-MRI), or Fluorescence Optical Imaging (FOI).

In another embodiment, an image enhancement agent may be substituted for the magnetic resonance image contrast enhancement agent or another image enhancement agent may be added to the magnetic resonance imaging contrast enhancement agent. The substituted or added image enhancement agent may be used with other medical imaging techniques. For example, a radionuclide may be substituted for or added to the magnetic resonance imaging contrast enhancement agent for use with Positron Emission Tomography. In another example, a fluorophore such as ICG, Cy7, or IR800 may be substituted for or added to the magnetic resonance imaging contrast enhancement agent for use with FOI.

In one or more embodiments of the invention, the magnetic resonance imaging contrast enhancement agent comprises particles including metal shells that are used to encapsulate imaging agents within an interstitial layer of dielectric to form a magnetic resonance imaging contrast enhancement agent. In one or more embodiments of the invention, the dielectric may be silica. In one or more embodiments of the invention, the overall dimension of the individual particles is less than 100 nm in diameter. In one or more embodiment, the metal shell is gold or silver.

In one or more embodiments, the particles comprising the magnetic resonance imaging contrast enhancement agent include a metal core within the interstitial layer of dielectric. When a metal core is present in the interstitial layer of dielectric, the particles include a metal core, an interstitial layer of dielectric, and a metal shell. The dielectric may be silica (SiO₂) or other dielectric material. The particle-in-shells design may be referred to as a nanomatryoshka. In one or more embodiments of the invention, the metal core is gold or silver. In one or more embodiments, the metal core may be rod shaped, star shaped, or a cube. In one embodiment of the invention, nanomatryoshkas may support plasmon resonances. A plasmon resonance is the collective oscillation of conduction band of electrons within a metal surface upon excitation with an external electromagnetic field. The plasmon resonance concentrates the external electromagnetic field to enhance the properties of contrast agents attached nearby the metal surfaces. The plasmon resonance of the metal core and the metal shell of nanomatryoshka particles may hybridize to give rise to a new hybrid mode, not present in particles that contain only include a metal shell. The new hybrid mode has a lower plasmon energy which causes a plasmon resonance between 200 nm and 1200 nm for nanomatryoshkas having a diameter of less than 100 nm. The nanomatryoshkas may have a large absorption cross-section that is tunable to a near-infrared laser (˜800 nm). Light absorbed near 800 nm is transduced to heat that can be efficiently used, for example, to destroy cancer cells in photothermal ablation therapy.

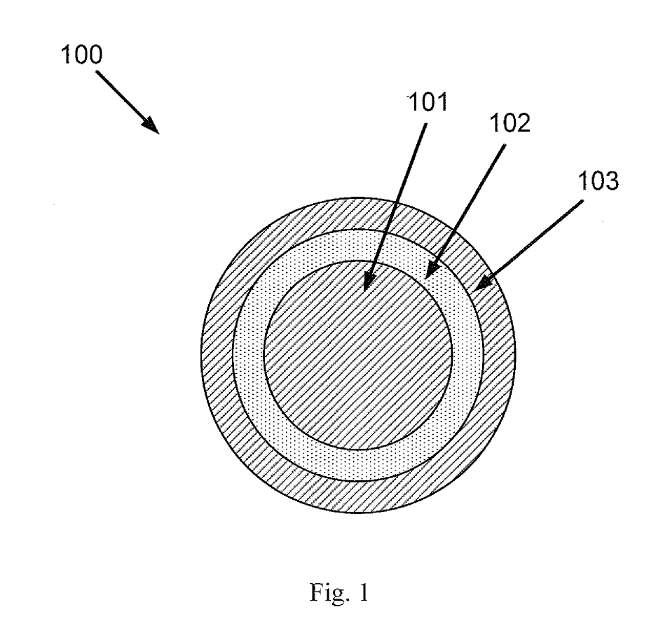

In accordance with one or more embodiments of the invention, FIG. 1 illustrates a cross sectional view of a single particle of a magnetic resonance imaging contrast enhancement agent (100). The particle includes a metal core (101), dielectric shell (102), and a metal shell (103). The dielectric shell (102) may be silica. The dielectric shell (102) may be other dielectric materials without departing from the invention.

In one or more embodiments, the dielectric shell is doped with 3-aminopropyl-triethoxysilane. The metal core (101) is approximately spherical in one or more embodiments. In further embodiments, the metal core (101) is rod shaped, star shaped, or a cube. The outer dimension of the metal core (101) is less than 40 nanometers. In one or more embodiments the metal core (101) is gold or silver.

The dielectric shell (102) is less than 20 nm thick and disposed on the metal particle (101). The dielectric shell (102) has a rough outer surface. The dielectric shell (102) is loaded with a solution of water and a type 1 contrast agent. A type 1 contrast agent appears brighter in a magnetic resonance imaging phantoms. In one or more embodiments, the dielectric shell (102) is loaded with a solution of water and a type 2 contrast agent. In one or more embodiments, the dielectric shell (102) is loaded with a solution of water, a type 1 contrast agent and a type 2 contrast agent. In one or more embodiments, the type 1 contrast agent is a lanthanide. In one or more embodiments, the lanthanide is gadolinium. In one or more embodiments, the type 1 contrast agent is manganese oxide and the type 2 contrast agent is iron oxide. In one or more embodiments of the invention, the type 1 contrast agent is chelated with diethylene triamine pentaacetic acid.

The metal shell (103) is disposed on the dielectric shell (102). The metal shell is between 1 and 20 nm thick and encapsulates the dielectric shell (102) and metal particle (101).

The radius of the metal particle (101), thickness of the dielectric shell (102), and thickness of the metal shell (103) are selected to support a plasmon resonance centered at greater than 400 nm and less than 1200 nm while keeping the total outer dimension of the particle below 100 nm. In one or more embodiments of the invention, the radius of the metal particle (101), thickness of the dielectric shell (102), and thickness of the metal shell (103) are selected to support a plasmon resonance centered at 810 nm while keeping the total outer dimension of the particle below 100 nm.

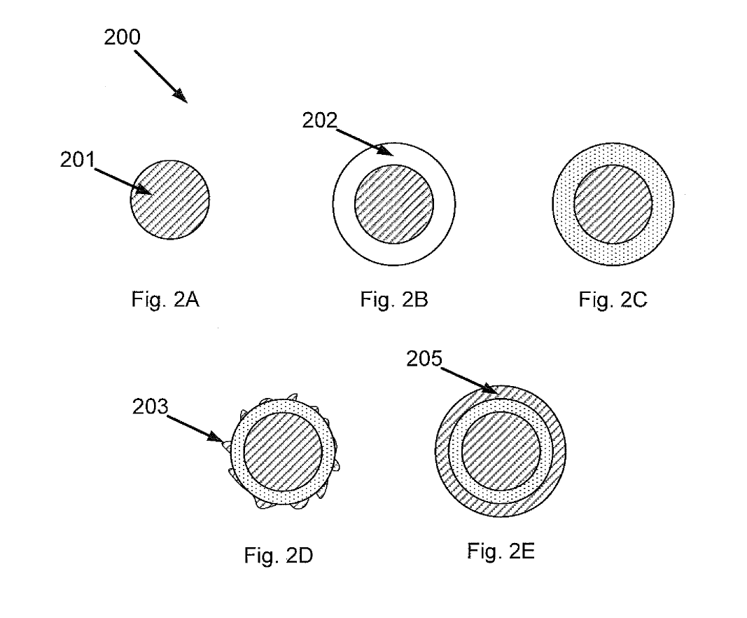

In accordance with one or more embodiments of the invention, FIG. 2 shows a series of panels illustrating a method of producing a magnetic resonance imaging enhancement agent (200). Each panel illustrates a cross sectional view of a part of the production method (200). One or more parts shown in FIG. 2 may be omitted, repeated, and/or performed in a different order among different embodiments of the invention. Accordingly, embodiments of the invention should not be considered limited to the specific number and arrangement shown in FIG. 2.

The method (200) starts with a metal core (201) as shown in panel (A). In Panel (A), the metal core is drawn as a circle but could be a cube, rod, or star shaped. A dielectric shell (202), shown in panel (B), is deposited onto the metal core (201) by a wet chemical process which is described below. Following the deposition of the dielectric shell (202), the dielectric shell (202) is submerged in a first solution that includes water and a type 1 contrast agent for a predetermined time. In one or more embodiments, the predetermined time is 14 hours. Other predetermined times may be used without departing from the invention. In one or more embodiments, the type 1 contrast agent is a lanthanide. In one or more embodiments, the lanthanide is gadolinium. In one or more embodiments, the type 1 contrast agent is manganese oxide or iron oxide.

Submerging the dielectric shell (202) results in water and a lanthanide being loaded into the dielectric shell (202) as illustrated in panel (C). The lanthanide may be chelated by diethylene triamine pentaacetic acid. Following submersion in the first solution, the dielectric shell (202) is submerged in a second solution including gold colloid produced through the reaction of sodium hydroxide, aqueous tetrakis(hydroxymethyl) phosphonium chloride, and aqueous chloroauric acid. The second solution both etches the dielectric shell (202) to reduce the thickness to a desired thickness and seed the dielectric shell (202) with metal as shown in panel (D). In one or more embodiments, the seeded metal (203) is silver or gold. Seeding the dielectric shell (202) results in small patches of metal (203) that are distributed over the dielectric shell (202) and adhered to the dielectric shell (202). Lastly, after etching and seeding the dielectric shell (202), a metal shell (205) is plated on the exterior surface of the dielectric shell (202) as shown in panel (E). In one or more embodiments, the metal shell is silver or gold.

In one embodiment of the invention, magnetic resonance imaging contrast enhancement agent is produced using a four step process including coating gold particles with APTES-doped dielectric, loading water and Gadolinium into the APTES-doped dielectric coating, etching the dielectric coating and seeding the dielectric coating with gold, and coating the dielectric coating with gold. Additional detail about the four step process is included below.

Step 1: Coating gold particles with APTES-doped silica. Gold colloid (50 nm citrate NanoXact Gold, nanoComposix) is coated with silica doped with 3-aminopropyl-triethoxysilane (APTES) by a modified Stöber process. (APTES is used as a binding site for gold colloid.) 21 mL of gold colloid (7.0×1010 particles/mL, citrate capped 50 nm Au sphere, NanoComposix) is added under stirring to an Erlenmeyer flask with a ground glass joint. Next, 180 mL of 200 proof ethanol (Decon Labs) and, 1.8 mL of ammonium hydroxide (28-30%, EMD Chemicals) are added to the gold colloid. Then, 36 μL of a solution of 10% tetraethoxysilane (TEOS, SIT7110.2, Gelest) in ethanol and 36 μL of 10% APTES (SIA0610.1, Gelest) in ethanol is added to the gold colloid. The gold colloid is sealed and stirred for 50 min at room temperature and then the gold colloid is cooled to 4° C. and stirred for 19 h. The gold colloid is transferred to a dialysis membrane (Spectra/Por 6, MWCO=10000, Spectrum Labs), previously washed with Milli-Q grade water to remove residual chemicals and then with ethanol to remove excess water. The gold colloid is then dialyzed in 1 gallon of 200 proof ethanol for at least 12 h at room temperature to remove ammonium hydroxide and the remaining free silanes (TEOS and APTES) from the gold colloid. The gold colloid is then cooled to 4° C. and centrifuged for 30 min at 2500 rcf to form a number of first pellets (the solution was centrifuged in aliquots of ˜15 mL using 50 mL plastic tubes). If the supernatant is still red, the centrifugation is repeated to recoup more particles in the form of additional first pellets.

Step 2: Loading water and Gadolinium into the APTES-doped silica coating. Disperse the first pellets by sonication in solution including a total volume of 7.5 mL of water solution, 10 mg/mL GdCl₃, and 15 mg/mL Gd-DTPA. Stir the solution of nanoparticles with gadolinium compounds for 14 h at room temperature. After stirring, the centrifuge the solution at 2000 rcf for 30 mins to form a second pellet. Then centrifuge the supernatant at 2500 rcf for 30 mins to form a third pellet. Disperse the second and third pellets in 700 μL of water, by sonicating for 30 seconds. Centrifuge the 700 μL of solution at 2000 rcf for 20 mins to form a fourth pellet. Disperse the fourth pellet in 1 mL of water by sonicating for 30 seconds to form an APTES-doped silica coated gold colloid.

Step 3: Etching the silica coating and seeding the silica coating with gold. First, synthesize a Duff colloid by quickly, under rapid stirring, adding 1.2 mL of 1 M NaOH to 180 mL of H₂O, followed by adding 4 mL of a 1.2 mM aqueous tetrakis(hydroxymethyl) phosphonium chloride (THPC, 80% solution in H₂O, Sigma). Stir for 5 min and then add 6.75 mL of 1% (w/v) aqueous chloroauric acid (HAuCl₄•3H₂O, Sigma-Aldrich). Refrigerate the solution for at least 2 weeks to form a Duff colloid.

Second, add 20 mL of Duff colloid to 50 mL plastic centrifuge tubes, followed by 300 μL of 1 M NaCl and 1 mL of APTES-doped silica coated gold colloid. Vortex and sonicate the solution for 10 min. After vortexing and sonicating, incubate the solution for 12 h at room temperature. Incubation in the solution etches and seeds with metal the APTES-doped silica coated colloid particles. After the incubation, sonicate the solution for 30 seconds and then centrifuged for 25 min at 900 rcf (10 mL in each tube) to form a number of fifth pellets. Disperse the fifth pellets in 800 μL of water by sonication for 1 min and transfer to several 2 mL centrifuge tubes. Centrifuge the solution for 20 min at 1100 rcf to form a series of sixth pellets. Disperse the sixth pellets in 1 mL of water by sonicating for 1 min to form an etched and seeded particle solution.

Step 4: Coating the etched and seeded particles with gold. A metal shell of gold around the etched and seeded particles was performed using a plating solution as a source of Au³⁺. A plating solution is prepared by mixing 200 mL of water, 50 mg of anhydrous potassium carbonate (K₂CO₃), and 3 mL of 1 wt % aqueous chloroauric gold solution followed by aging for 12-19 h. The reduction of Au³⁺ into a metal shell of Au around the silica coating is performed in a 4.5 mL methacrylate cuvette with a plastic cap. Add a volume of 1.5 mL of plating solution to the cuvette followed by 20-60 μL of the etched and seeded particle solution. Next, add 7.5 μL of formaldehyde dropwise inside the cap, and close the cuvette. Shake the cuvette containing the solution for about 1 min to complete the plating process. The formaldehyde initiated the plating process by reduced the gold ions included in the plating solution.

Examples of magnetic resonance imagining contrast enhancement agents in accordance with one or more embodiments were characterized using a transmission electron microscope.

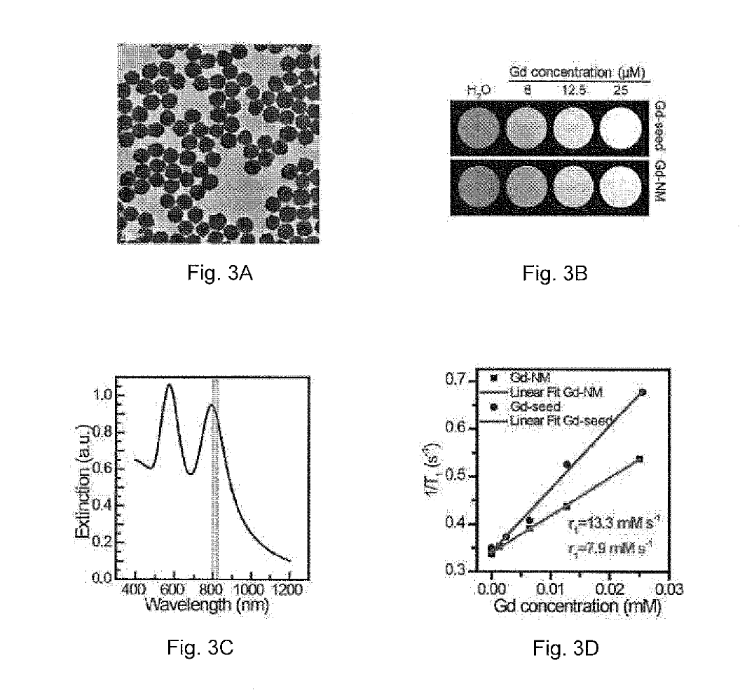

FIG. 3(A) shows a transmission electron microscope image of the magnetic resonance imaging contrast enhancement agent in accordance with one or more embodiments of the invention. The magnetic resonance imaging contrast enhancement agent is mono-dispersed in size and the outer dimension of each particle is less than 100 nm.

FIG. 3(B) shows extinction spectra of a magnetic resonance imaging contrast enhancement agent in accordance with one or more embodiments of the invention. The plasmon resonance of the magnetic resonance imaging contrast enhancement agent has been tuned to 810 nm as indicated in FIG. 3(B).

FIG. 3(C) shows the T1 enhancement of a magnetic resonance imaging contrast enhancement agent in accordance with one or more embodiments of the invention (labeled as Gd-seed) and Gadolinium-DTPA (labeled as Gd-NM). The magnetic resonance imaging contrast enhancement agent provides superior T1 enhancement to Gadolinium-DTPA for all levels of gadolinium concentration as indicated by the color of each circle in the plot.

FIG. 3(D) shows the relaxivity of a magnetic resonance imaging contrast enhancement agent in accordance with one or more embodiments of the invention and Gadolinium-DTPA. As seen, the magnetic resonance imaging contrast enhancement agent provide superior relaxivity over Gadolinium-DTPA for all levels of concentration as indicated by the greater slope of the magnetic resonance imaging contrast enhancement agent.

While the invention has been described with respect to a limited number of embodiments, those skilled in the art, having the benefit of this disclosure, will appreciate that other embodiments can be devised which do not depart from the scope of the invention as disclosed herein. Accordingly, the scope of the invention should be limited only by the attached claims.

What is claimed is:

1. A Magnetic Resonance Imaging (MRI) enhancement agent, comprising:
a plurality of particles, each particle comprising:
a metal core;
a dielectric shell disposed on the metal core comprising water and at least one MRI contrast agent; and
a metal shell disposed on the exterior surface of the dielectric shell that encapsulates the dielectric shell.

2. The magnetic resonance imaging enhancement agent of claim 1, wherein a radius of the metal core is less than 40 nm.

3. The magnetic resonance imaging enhancement agent of claim 1, wherein a radius of the metal core is approximately 25 nm.

4. The magnetic resonance imaging enhancement agent of claim 1, wherein a radius of the metal core is less than 25 nm.

5. The magnetic resonance imaging enhancement agent of claim 1, wherein a thickness of the silica shell is less than 20 nm.

6. The magnetic resonance imaging enhancement agent of claim 1, wherein a thickness of the metal shell is between 1 and 20 nm.

7. The magnetic resonance imaging enhancement agent of claim 1, wherein at least a portion of the plurality of particles supports a plasmon resonance centered at greater than 400 nm and less than 1200 nm.

8. The magnetic resonance imaging enhancement agent of claim 1, wherein at least a portion of the plurality of particles supports a plasmon resonance centered at 810 nm.

9. The magnetic resonance imaging enhancement agent of claim 1, wherein the silica shell is doped with an amine.

10. The magnetic resonance imaging enhancement agent of claim 9, wherein the amine is 3-aminopropyl-triethoxysilane.

11. The MRI enhancement agent of claim 1, wherein the MRI contrast agent is comprised of at least one selected from the group containing a type 1 contrast agent and a type 2 contrast agent.

12. The magnetic resonance imaging enhancement agent of claim 11, wherein the type 1 contrast agent is manganese oxide and the type 2 contrast agent is iron oxide.

13. The magnetic resonance imaging enhancement agent of claim 11, wherein the type 1 contrast agent is a lanthanide.

14. The magnetic resonance imaging enhancement agent of claim 13, wherein the type 1 contrast agent is chelated.

15. The magnetic resonance imaging enhancement agent of claim 13, wherein the lanthanide is gadolinium.

16. The magnetic resonance imaging enhancement agent of claim 14, wherein the chelate is diethylene triamine pentaacetic acid.

17. The magnetic resonance imaging enhancement agent of claim 1, wherein the magnetic resonance imaging enhancement agent is comprised of at least one type 1 contrast agent and at least one type 2 contrast agent.

18. The magnetic resonance imaging enhancement agent of claim 17, wherein the type 1 contrast agent is a lanthanide.

19. The magnetic resonance imaging enhancement agent of claim 18, wherein the lanthanide is gadolinium.

20. The magnetic resonance imaging enhancement agent of claim 17, wherein the type 1 contrast agent is manganese oxide and the type 2 contrast agent is iron oxide.

21. The magnetic resonance imaging enhancement agent of claim 1, wherein the metal core is gold.

22. The magnetic resonance imaging enhancement agent of claim 1, wherein the metal shell is gold.

23. The magnetic resonance imaging enhancement agent of claim 1, wherein the metal core is silver.

24. The magnetic resonance imaging enhancement agent of claim 1, wherein the metal shell is silver.

25. A method of producing a Magnetic Resonance Imaging (MRI) enhancement particle, comprising:
coating a metal core with a dielectric to obtain a metal core with a dielectric coating;
loading the dielectric coating with a solution comprising water and a MRI contrast agent to obtain a loaded dielectric coating;
etching the loaded dielectric coating to a desired thickness to obtain an etched dielectric coating;
seeding the exterior of the etched dielectric coating with a metal to obtain a seeded dielectric coating; and
coating the seeded dielectric coating with a second metal to obtain the magnetic resonance imaging enhancement particle.

26. The method of claim 25, wherein the dielectric shell is doped with an amine.

27. The method of claim 26, wherein the amine is 3-aminopropyl-triethoxysilane.

28. The method of claim 25, wherein the MRI contrast agent is comprised of at least one selected from the group containing a type 1 contrast agent and a type 2 contrast agent.

29. The method of claim 28, wherein the type 1 contrast agent is a lanthanide.

30. The method of claim 29, wherein the lanthanide is gadolinium.

31. The method of claim 25, wherein the type 1 contrast agent is chelated.

32. The method of claim 31, wherein the chelate is diethylene triamine pentaacetic acid.

33. The method of claim 28, wherein the type 1 contrast agent is manganese oxide and the type 2 contrast agent is iron oxide.

34. The method of claim 25, wherein the MRI contrast agent is comprised of at least one type 1 contrast agent and at least one type 2 contrast agent.

35. The method of claim 32, wherein the type 1 contrast agent is a lanthanide.

36. The method of claim 33, wherein the lanthanide is gadolinium.

37. The method of claim 32, wherein the type 1 contrast agent is manganese oxide and the type 2 contrast agent is iron oxide.

38. The method of claim 25, wherein the metal core is gold.

39. The method of claim 25, wherein the metal core is silver.

40. The method of claim 25, wherein the second metal is silver.

41. The method of claim 25, wherein the second metal is gold.

42. The method of claim 25, wherein the dielectric shell is silica.
